(19) United States
(12) Patent Application Publication
(10) Pub. No.: US 2008/0031903 A1
(43) Pub. Date: Feb. 7, 2008

(54) METHOD OF TREATING OCULAR INFECTIONS

(76) Inventors: Andrea Gambotto, Pittsburgh, PA (US); Edward Chieke Nwanegbo, Pittsburgh, PA (US)

Correspondence Address: JESSE A. HIRSHMAN, ESQ., 1722 MURRAY AVENUE, THIRD FLOOR, PITTSBURGH, PA, 15217, US

(21) Appl. No.: 11/828,542

(22) Filed: Jul. 26, 2007

Related U.S. Application Data
(60) Provisional application No. 60/833,568, filed on Jul. 27, 2006.

(51) Int. Cl. A61K 39/395; A61K 31/13; A61K 31/135; A61K 31/165; A61P 31/00; A61P 33/00; A61K 9/00; A61K 31/40; A61K 31/497

(52) U.S. Cl. 424/400; 424/130.1; 424/133.1; 424/147.1; 424/150.1; 424/151.1; 424/152.1; 424/159.1; 424/164.1; 514/253.06; 514/412; 514/619; 514/648; 514/669

(57) ABSTRACT

Provided herein are methods of preventing or treating ocular viral, fungal, protozoa or bacterial infections. The method comprises administering ocular doses of a binding agent reactive against a virus, fungus, protozoa or bacteria, which, in one embodiment, is a pooled, human immunoglobulin preparation, such as, without limitation, those suitable for intravenous use, including Gammagard or other like products. Also provided are ocular dosage forms and dosing devices comprising the binding reagent.

### CROSS REFERENCE TO RELATED APPLICATIONS

This application claims the benefit under 35 U.S.C. §119(e) to United States Provisional Patent Application No. 60/833,568, filed on Jul. 27, 2006, which is incorporated herein by reference in its entirety.

Described herein are methods of treating ocular infections and related compositions and products. The methods comprise administering to an eye of the patient a composition comprising a binding reagent reactive against the viral, fungal, protozoa or bacterial agent in an amount effective to prevent or treat the infection.

Epidemic Keratoconjuctivitis (EKC) is a debilitating infectious disease of the eye that is seen all over the world. The disease is caused mostly by adenoviruses especially serotype 8, 19 and 37. Serotype 3, 4 and 11 were also implicated in some EKC epidemics. The disease affects all age groups, is highly contagious and spreads quickly in schools, schools, swimming pools, pediatric unit and camps. Treatment is presently symptomatic as there is no effective treatment. Development of effective anti-viral topical agent is desirable to treat the disease and prevent epidemic.

Conjunctivitis also can be caused by a number of additional bacterial, viral, fungal and protozoa agents, including, but not limited to: S. aureus, S. pneumoniae, H. influenzae, Neisseria gonorrhoeae and Chlamydia trachomatis, Adenovirus, Herpes Simplex, Herpes zoster virus, Enteroviruses, Fusarium species, Candida species and Acanthamoeba species. Certain viral infections, such as adenoviral infections may be treated with antiviral drug products, such as cidofovir. Typically, drug products have side effects, such as the ocular and renal side effects associated with cidofovir. Other logistical issues arise with drug products, including stability, cost of production, etc. As such, an inexpensive, readily-available, well-accepted and stable drug product for treatment of ocular infections is desirable.

### SUMMARY

Described herein are methods, compositions and drug products for prevention and treatment of ocular infections. Specifically, it has been found that ocular administration of binding reagents, such as, without limitation, antibody preparations, including pooled immunoglobulin preparations (compositions comprising pooled immunoglobulin), are effective in treating certain ocular infections. Thus, a method is provided comprising administering to an eye of the patient a composition comprising a binding reagent reactive against a viral, fungal, protozoa or bacterial agent in an amount effective to prevent or treat the infection. In one non-limiting embodiment, the binding reagent is an antibody, such as a monoclonal antibody or a polyclonal antibody, such as a human or humanized antibody. In other non-limiting embodiments, the composition comprises a binding reagent comprising directed or non-directed human immunoglobulin, a pooled human immunoglobulin preparation, or a pooled human IG preparation, such as, without limitation, Gammagard S/D immune Globulin Intravenous (Human) (Baxter Healthcare Corporation).

In one non-limiting embodiment, the viral, fungal, protozoa or bacterial agent is an adenovirus. For treatment of ocular infections, without limitation, between 1 μg and 100 mg of pooled human immunoglobulin is administered to the patient from one to ten times daily for a suitable time period, for example and without limitation, from one day to two months, from 3 to 21 days, from 7 to 14 days, or for 14 days. In another non-limiting embodiment, for treatment of adenovirus, about 10 μg to 5 mg of pooled human IG is administered from two to six times daily for one to 14 days. In other non-limiting embodiments, the viral, fungal, protozoa or bacterial agent is one of S. aureus, S. pneumoniae, H. influenzae, Neisseria gonorrhoeae and Chlamydia trachomatis, Adenovirus, Herpes Simplex, Herpes zoster virus, Candida species, Acanthamoeba species and Enteroviruses, and the binding reagent is administered in an amount effective to treat or prevent infection by the agent.

In yet another non-limiting embodiment, an anti-inflammatory agent is administered while the binding reagent is administered to the patient, and may be co-administered with the binding reagent in a single drug product (composition or preparation) containing both the binding reagent and the anti-inflammatory agent. The anti-inflammatory agent may be a non-steroidal anti-inflammatory agent, such as, without limitation, one or more of nepafenac, ketorolac, tromethamine, acetaminophen and bromfenac. In another non-limiting embodiment, an antibiotic is administered while the binding reagent is administered to the patient, and may be co-administered with the binding reagent and, optionally, an anti-inflammatory agent, in a single drug product (composition or preparation) containing both the binding reagent, the antibiotic, and, optionally, the anti-inflammatory agent. Non-limiting examples of suitable antibiotics include: ciprofloxacin, norfloxacin, afloxacin, levofloxacin, gentamicin, tobramycin, neomycin, erythromycin, trimethoprim sulphate, and polymixin B.

The binding reagent, along with any other active agents, typically is administered in an opthamologically acceptable carrier, which may be a liquid or hydrogel. The composition may comprise one or more of CMC (carboxymethylcellulose), PVP (polyvinylpyrrolidone), a buffer, a rheology modifier (thickening agent), a buffer and a chelating agent.

Also provided is a composition comprising a binding reagent reactive against a viral, fungal, protozoa or bacterial agent and an anti-inflammatory agent and/or an antibiotic in amounts effective to treat an ocular infection by the bacterial agent an opthamologically acceptable carrier. The binding reagent and anti-inflammatory agent and/or antibiotic may be, without limitation, a binding reagent, anti-inflammatory agent or antibiotic as described above and elsewhere in this document in the context of the methods described herein.

In the implementation of the methods described herein and useful in commercial distribution and effective dosing of the product, a product is provided that comprises an opthamologically-acceptable ocular dispenser containing a composition comprising a binding reagent reactive against a viral, fungal, protozoa or bacterial agent in amounts effective to treat an ocular infection by the viral, fungal, protozoa or bacterial agent in an opthamologically acceptable carrier. The composition optionally comprises an anti-inflammatory agent and/or an antibiotic. The opthamologically acceptable ocular dispenser may be, without limitation, an eye-dropper or an eye cup, as are broadly known in the art. The binding reagent and anti-inflammatory compound and/or antibiotic may be, without limitation, a binding reagent, anti-inflammatory compound or antibiotic as described above and elsewhere in this document in the context of the methods described herein.

### BRIEF DESCRIPTION OF THE DRAWINGS

FIG. 1 is a graph showing flow cytometric comparison of neutralization of Ad5EGFP by two batches of pooled human IG.

FIG. 2 is a photograph of a sample microneutralization described in Example 2.

FIG. 3 is a graph showing the results of the microneutralization assay described in Example 2 in A549 cells.

FIG. 4 is a graph showing the results of the microneutralization assay described in Example 2 in HeLa cells.

FIG. 5 is a graph showing the results of the microneutralization assay described in Example 2 in conjunctiva cells.

FIG. 6 summarizes the results of number of positive cultures per total during the study period (day 1-14 post infection) as described below in the Examples.

FIG. 7 is a graph showing ocular viral shedding in early phase (day 1-5) and late Phase (day 7-14) in rabbits, as described below in the Examples.

FIG. 8 is a graph showing the duration of viral shedding in rabbits for the three treatment groups described in the Examples below.

FIG. 9 is a graph showing daily ocular viral titers in rabbits for the three groups described in the Examples below.

FIG. 10 is a graph showing the mean combined ocular viral titers in rabbits for the three groups described in the Examples below.

FIG. 11 is a graph showing 1 Log 10 reduction in titer by neutralization of multiple clinical ocular isolates of Adenoviral serotypes. This figure presents the IG concentration that decreases titers of multiple clinical ocular isolates of adenoviral serotypes and ATCC type Ad37 by 1 log 10 pfu/ml. The numbers in the x-axis labels represent the adenovirus serotype, while the letters represent multiple isolates of the same serotypes.

FIG. 12 is a graph demonstrating the percent Ad5 positive cultures per total for each culture day for eyes treated with 100 mg/ml Human IG (IG) (●) (n=20), 0.5% Cidofovir (X) (n=20) and Saline (□) (n=20). Swabs were taken on Days 0, 1, 3, 4, 5, 7, 9, 11 and 14 post-infection. Both 100 mg/ml or IG and 0.5% Cidofovir demonstrated significantly fewer Ad5 positive cultures per total compared with the Saline control on Days 7 and 9 (Chi-Square). There were no significant differences among the groups on any other day.

FIGS. 13A and B show the Mean Ad5 Ocular Daily Titers for the data presented in Examples 3 and 5. Animals were infected with Ad5 and treated with study drugs. Serial ocular viral cultures were carried out and mean daily viral ocular titers calculated. The combined Mean Daily Ocular titers of the two studies during early (FIG. 13A, days 1-5) and late (FIG. 13B, days 7-14) phases of infection in animals treated with topical IG (●) was compared with animals treated with Saline (□) and Cidofovir (X). The bar indicates SEM of the two studies. (*Topical IG significantly reduced Mean Daily Ocular titers on these days compared to the Saline treated animals)

FIG. 14 is a graph showing in vitro inhibition of HSV1 by Human IG.

### DETAILED DESCRIPTION

Provided herein are methods of treating ocular infections, particularly conjunctivitis and EKC. As used herein, a binding reagent-containing preparation, such as, without limitation, a pooled human immunoglobulin preparation, is shown to be useful in treating bacterial, viral, fungal and protozoa ocular infections. As such a method of treating an ocular infection is provided. The method is useful in treating ocular adenoviral infections as well as for treating infections caused by other bacterial, fungal, protozoa and viral etiological agents, including, without limitation: Staphylococcus aureus, Streptococcus pneumoniae, Haemophilus influenzae, Neisseria gonorrhoeae and Chlamydia trachomatis, Pseudomonas aeruginosa, Adenovirus, Herpes Simplex, Herpes zoster virus, Fusarium species, Candida species, Acanthamoeba species and Enteroviruses. To implement the described methods, also provided are compositions and products comprising an ocular drug dispenser containing a binding reagent specific to an ocular infectious agent, such as pooled human immunoglobulin or a pooled human immunoglobulin preparation that is suitable for intravenous use.

As used herein, “pooled human immunoglobulin” (Immune globulin) is immunoglobulin obtained from two and preferably more individuals. In one embodiment, the pooled human immunoglobulin is predominantly a preparation of pooled human immunoglobulin, such as pooled human immunoglobulin that is suitable for intravenous use (approved for intravenous use, equivalent to a product approved for intravenous use or medically or pharmaceutically acceptable for intravenous use), referred to herein, without limitation, as “IG,” which is available commercially, for example and without limitation as Gammagard® S/D Immune Globulin Intravenous, commercially available from Baxter Healthcare Corporation of Westlake Village, Calif., which is prepared from large pools of human plasma by Cohn-Oncley cold ethanol fractionation followed by ultrafiltration and ethanol fractionation. As can be appreciated by one of skill in the art, any polyvalent antibody-containing fraction obtained from more than one individual, and preferable more than 5, 10, 20, 25 or even 50 individuals would suffice. The Gammagard® S/D manufacturing process provides a product that demonstrates a significant viral reduction in in vitro studies. These studies demonstrate virus clearance during Gammagard® S/D manufacturing using infectious Human Immunodeficiency virus, Types 1 and 2 (HIV-1, HIV-2); Sindbis virus (SIN), a model virus for Hepatitis C virus; Pseudorabies virus (PRy), a model virus for lipid-enveloped DNA viruses such as Herpes; and Vesicular stomatitis virus (VSV), a model virus for lipid-enveloped RNA viruses. As such, though not tested in an ocular environment, a pooled multivalent immunoglobulin preparation such as Gammagardφ S/D is reactive against many enveloped and non-enveloped viruses. IG (Gammagard® S/D) is a known clinical product that has been approved for the treatment for Idiopathic Thrombocytopenic purpura, Kawasaki syndrome and Chronic lymphocitic Leukemia (CLL). It has broad spectrum anti-microbial properties. Because of this, it has also been used in life threatening sepsis. Methods of making pooled immunoglobulin, such as pooled human immunoglobulin are well known in the art, for example and without limitation, as described in U.S. Pat. Nos. 6,281,336 and 5,965,130, both of which are hereby incorporated herein by reference in their entirety.

Other binding reagent preparations, e.g. antibody preparations, such as polyvalent immune serum or even monoclonal antibodies or recombinant binding reagents may be used in place of, or in addition to the pooled human polyvalent immunoglobulin, but typically at much greater expense. These binding reagents are “directed,” meaning, in these examples, that their donors were immunized to elicit a target-antigen-specific humoral response. Preferably the antibody or other binding reagent is of human origin or “humanized” as is known in the art.

The term “binding reagent” and like terms, refers to any compound, composition or molecule capable of specifically or substantially specifically (that is with limited cross-reactivity) binding another compound or molecule, which, in the case of immune-recognition contains an epitope. In many instances, the binding reagents are antibodies, such as polyclonal or monoclonal antibodies. “Binding reagents” also include derivatives or analogs of antibodies, including without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)fragments), diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific or non-specific binding reagent compositions, including antibodies and their derivatives and analogs, are well-known in the art. Directed polyclonal antibodies can be generated by immunization of an animal and recovery of plasma. Pooled polyclonal antibodies are obtained from multiple subjects, including humans, which may be directed (each subject is vaccinated with a specific antigen, as in the common case of production of polyclonal antibodies in animal subjects, such as rabbits or horses) or non-directed (subjects are not specifically immunized with an antigen, as in the case of Gammagard® S/D). Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from GE Healthcare Bio-Sciences Corp. of Piscataway, N.J. and the pSKAN Phagemid Display System, commercially available from MoBiTec GmbH, of Goettingen Germany. Aptamer technology is described for example and without limitation in U.S. Pat. Nos. 5,270,163, 5,475,096, 5,840,867 and 6,544,776.

In one embodiment, the binding reagent blocks infectivity of the pathogen and therefore is directed to an antigen of the pathogen responsible for, for example and without limitation, adsorption, binding and/or virulence. In an alternative embodiment, the binding reagent interferes with any aspect of the viral or pathogen life cycle. This reagent apart from pathogen clearance activity, can potentially prevent formation of sub-epithelial infiltrate because of its anti-inflammatory and immune-modulating properties. A binding reagent is said to be “reactive against” a pathogen (e.g., virus, fungus, protozoa or bacteria) inasmuch as it neutralizes or otherwise interferes with infection, growth, propagation, virulence, spreading, dissemination or any other activity of the pathogen that contributes to infection and dissemination of the pathogen in individuals or populations of individuals.

In addition to the binding reagent-containing composition, an anti-inflammatory agent may be co-administered in an amount effective to augment decrease of ocular inflammation and pain associated with a given infection. Steroidal anti-inflammatories are useful, but not preferred because they cause corneal thinning and prolong viral shedding. Non-steroidal anti-inflammatories (NSAIDs) suitable for ocular use are preferred and include, without limitation: nepafenac (for example and without limitation, Nevenac 0.1%, nepafenac ophthalmic suspension, Alcon Laboratories, Inc.), ketorolac tromethamine (for example and without limitation, Acular LS 0.4%, ketorolac tromethamine ophthalmic suspension, Allergan, Inc.), acetaminophen and bromfenac (for example and without limitation, Xibrom 0.09%, bromfenac ophthalmic suspension, Ista Pharmaceuticals). Thus, also provided herein is a drug product comprising both a binding reagent and a pharmaceutically acceptable anti-inflammatory suitable for optical use. These anti-inflammatory compounds often exhibit analgesic effects. In any case, according to the methods described herein, the binding reagent and the anti-inflammatory may be contained in the same composition, but also may be administered separately in a manner effective to treat the infection.

In one non-limiting embodiment, an antibiotic also may be co-administered along with the binding reagent and, optionally, the anti-inflammatory agent may also be co-administered with the binding reagent and the antibiotic, all in an amount effective to treat and/or prevent infection. Non-limiting examples of suitable antibiotics include: ciprofloxacin, norfloxacin, afloxacin, levofloxacin, gentamicin, tobramycin, neomycin, erythromycin, trimethoprim sulphate, and polymixin B.

In any case, as used herein, any agent used for prevention or treatment of an ocular infection is administered in an amount effective to treat or prevent that infection, namely in an amount and in a dosage regimen effective to prevent, reduce the duration and/or severity of the infection and/or shedding of the infectious agent. As shown herein, 37 μl of a 100 mg/ml solution of human pooled immunoglobulin (IG) was administered four times daily in one drop per eye to achieve effective treatment of an adenoviral infection in rabbits. Different concentrations of immunoglobulin and different dosage regimens will achieve similar results, with the drug product administered, typically and without limitation, from one to ten times daily, including 2, 3, 4, 5, 6, 7, 8, 9 and 10 times daily. The amount (e.g., number of drops of drug product) of the drug product administered to the patient (typically one or two drops per eye per dose when a dropper is used), also may vary depending on the ocular dispenser used to administer the drug product and the concentration of the binding reagent and, where appropriate, anti-inflammatory agent in the drug product. A person of average skill in the pharmaceutical and medical arts will appreciate that it will be a matter of simple design choice and optimization to identify a suitable dosage regimen for treatment of any given ocular infection or prevention of an ocular infection. The amount of binding reagent administered also will affect outcome. A range of from about 10 μg to about 100 mg of pooled human immunoglobulin, for example about 500 μg, may be administered per dose.

Ocular dosage forms include, without limitation, eye drops (liquids), ointments, oils, multi-phase systems (such as, liposome, micellular, homogenates or suspensions of liquids or semi-solid or solid particles), gels, creams, pads or strips. In one embodiment, the active ingredient (drug) is in a water-based (aqueous) drug product. In another embodiment, the active ingredient is in a petrolatum-based drug product. One embodiment of the present invention is the use of topical formulations of binding reagents as described herein to treat ocular infections caused by, without limitation, adenovirus. In one embodiment, a combined dosage form is provided comprising pooled human immunoglobulin in combination with a second or third active ingredient, such as, without limitation, an anti-inflammatory agent and/or an antibiotic. The dosage form comprises an opthamologically carrier which comprises acceptable excipients, such as, without limitation, one or more suitable: vehicle(s), solvent(s), diluent(s), pH modifier(s), buffer(s), salt(s), colorant(s), rheology modifier(s), lubricant(s), filler(s), antifoaming agent(s), erodeable polymer(s), hydrogel(s), surfactant(s), emulsifier(s), adjuvant(s), preservative(s), phospholipid(s), fatty acid(s), mono-, di- and tri-glyceride(s) and derivatives thereof, wax(es), oil(s) and water, as are broadly known in the pharmaceutical arts

Also provided herein is a product comprising an ocular drug dispenser containing and, therefore, for delivery of a binding reagent as described herein, for example and without limitation, a pooled human IG preparation, such as Gammagard® S/D, optionally also containing an anti-inflammatory agent, as described above. A suitable ocular drug dispenser typically is an eye dropper, which typically is a squeezable vial (container) with an integral dropper tip. As is well, known in the art, the structure of the dropper tip, as well as the overall composition of the liquid or hydrogel drug product determines drop size and therefore the dosage regimen appropriate for that dispenser. In another embodiment, the ocular dispenser is an eye cup, facilitating washing of the eye and full contact with a solution. Suitable ocular dispensers are broadly available in the pharmaceutical industry from a variety of specialty manufacturers, and non-limiting examples of which are described in U.S. Pat. Nos. 6,814,265, 6,336,571, 5,582,330, 5,108,007, 5,048,727 and 5,033,647, each of which are incorporated herein by reference in their entirety. Further, a survey of commercially-available ocular drug products on the shelves of the average pharmacy illustrates many of the variations such dispensers can take. Of course, the eye dropper per se need not be integral with the vial, but it is preferable for control of product sterility. In any case, an ocular drug dispenser is a device useful and acceptable in the pharmaceutical arts for the controlled delivery of a drug product to the eye.

### EXAMPLES

### Example 1

The inhibition of infection of human epithelial cells by EKC causative Adenoviral serotypes was studied in vitro using serially diluted clinical grade Intravenous Immunoglobulin G (IG). 5×10⁸ viral particle of wild-type Adenoviral serotype 3, 4, 8, 11, 19 and 37 were incubated with serially diluted IG for 1 hour in 96 well plate. Freshly harvested cells of A549 cell line, HeLa cell line or Conjuctiva cell line were seeded and incubated for 72 hours at 37° C. Prevention of cell infection was analyzed using crystal violet-formaldehyde staining. Similar experiment was conducted using recombinant adenoviral serotype 5 encoding EGFP, but analyzed by flowcytometry after 24 hours incubation. Concentration of IG required to prevent cell infection by EKC serotypes was determined. Using the above technique, we found that Adenoviral serotype 5, 3, 4, 8, 11, 19 and 37 were prevented from infecting cell lines by low IG dilution. Less than 0.7 mg of IG is required to prevent infection of all three epithelial cells by EKC causative viruses. This finding indicates that a solution containing 5 mg/ml of IG administered topically in divided doses to patients suffering from EKC may abolish the infection, prevent complication, reduce viral shedding and prevent transmission of the disease. This treatment may cost less than $0.25 US and will be affordable in developing countries where EKC is endemic.

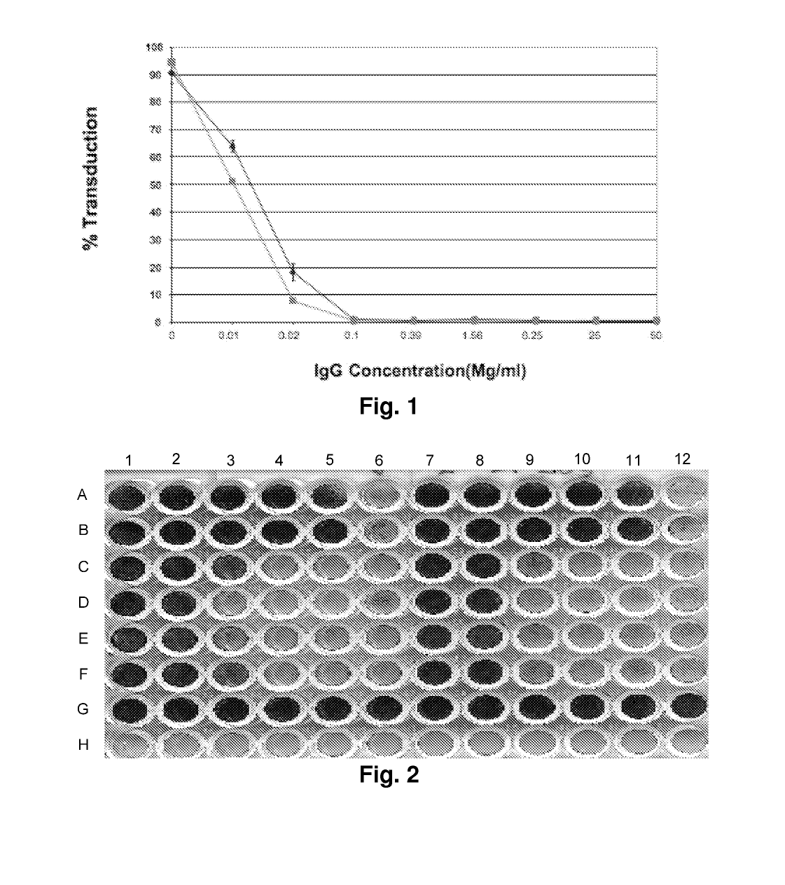

In the experiment employing recombinant adenoviral serotype 5 encoding enhanced green florescent protein (Ad5EGFP), Ad5EGFP was incubated with serially diluted IG for one 1 hour at 37° C. in 96 well plate. Freshly harvested A549 cells (10⁵) were seeded to the wells and incubated overnight. Results were analyzed by flow cytometry. The assay was repeated with another batch of IG. As shown in FIG. 1, very low concentrations of IG prevented Ad5 transduction of A549 cell line.

### Example 2

To study the effect of IG on wild type viruses, a microneutralization assay was developed. After optimization, 5000 viral particles per cell (total is 5×10⁸ viral particles) of wild type EKC causative adenoviral serotypes were incubated with serial dilutions of Iv-IgG in 96 well flat bottom plate for 1 hour at 37° C. in duplicate. 10⁵ cells of A549 cell line, HeLa cell line or Conjunctiva cell line was seeded to the wells and incubated for 72 hours at 37° C. Plates were washed after incubation and stained with a Crystal violet-formaldehyde solution. Controls included wells with only cell and IG, wells with cells and viral serotype, wells with media only. Infectivity of the cell line is indicated by absence of staining in wells containing only cell and the study viral serotype. The plate appearance after the assay is shown in FIG. 2. Wells of rows A to F contain all the test viruses. In Row G, only cells were seeded to the wells. Only media was added in all the wells in Row H. Serial IG dilution was carried out from column 1 and 7. Column 6 and 12 received no IG dilution and evaluates viral infectivity of the cell line. In this assay, all serotypes infected A549 cell line. This was demonstrated in the absence of staining in column 6 and 12. As shown in FIG. 2, the IG dilution at which cell staining occurred is the concentration of IG that prevented viral infection of the cell.

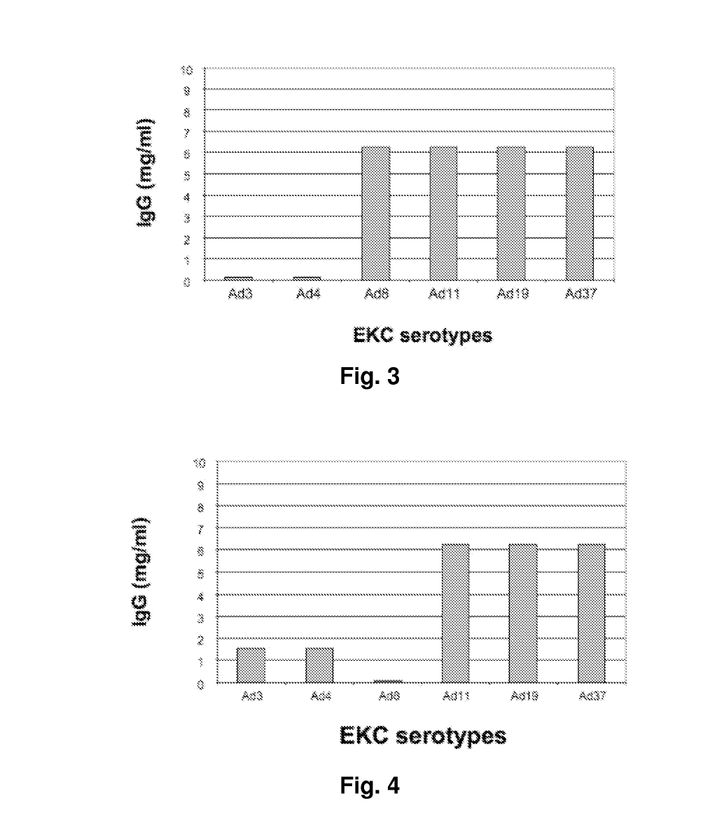

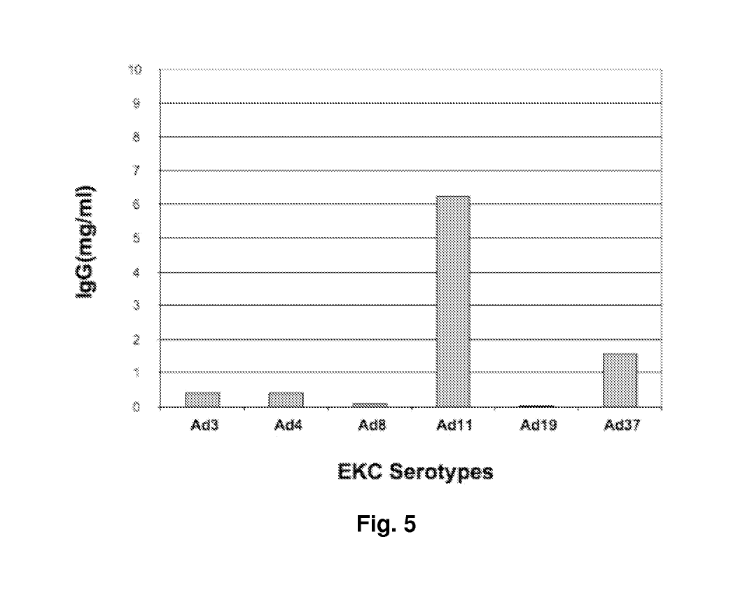

Using this method it was found that 625 μg of IG prevented infection by Ad8, Ad11, Ad19 and Ad37 in A549 cell line. Ten μg prevented infection by Ad3 and Ad4. See FIG. 3. In HeLa, cells, 625 μg of IG prevented infection by adenoviral serotype 11, 19 and 37. 156 μg prevented infection by Adenoviral serotype 3 and 4. See FIG. 4. In the conjunctiva cell line, 625 μg prevented infection of Ad11, while less than 200 μg was required to prevent infection by Adenoviral serotype 3, 4, 8, and 37. Ad19 displayed poor infectivity in conjunctiva cells. See FIG. 5.

These findings indicate that less than 1 mg of human IG pooled, polyvalent, non-directed human IG is required for broad-spectrum antiviral activity against most of the implicated EKC causative adenoviral serotypes. See Table 1. For example and without limitation, a solution containing 5 mg/ml of IG may be administered topically in 100 μl per drop two to three times daily and may be effective against Epidemic Keratoconjuctivitis. As a result this treatment would be highly cost-effective.

TABLE 1
Summary: IG (Mg/ml) Neutralization of EKC serotypes

| EKC Viruses | HeLa | A549 | CJ | Mean |
|---|---|---|---|---|
| Ad3 | 1.56 | 0.1 | 0.4 | 0.686667 |
| Ad4 | 1.56 | 0.1 | 0.4 | 0.686667 |
| Ad8 | 0.1 | 6.25 | 0.1 | 2.15 |
| Ad11 | 6.25 | 6.25 | 6.25 | 6.25 |
| Ad19 | 6.25 | 6.25 | 0.03 | 4.176667 |
| Ad37 | 6.25 | 6.25 | 1.56 | 4.686667 |

In this study, a high amount of virus (5×10⁶ pfu or 5×10⁸ viral particles) was used, which is unlikely to be the case in human clinical infection. Therefore the minimal concentration of IG effective in topical solution should be less than the minimal concentration effective in vitro.

### Example 3

### In Vivo Study

The purpose of this study is to determine the antiviral efficacy of a 100 mg/ml solution of human Intra Venous Immune Globulin (IG) in the Ad5/NZW rabbit ocular model.

Fifteen NZW female rabbits 2-3 lbs were received from Myrtle's Rabbitry, Thompson Station, Tenn. Fifteen rabbits were inoculated in both eyes following general anesthesia with ketamine & xylazine, topical anesthesia with proparacaine, and corneal scarification (12 cross-hatched strokes of a #25 needle) with 50 μl of 3.0×10⁷ pfu/ml (1.5×10⁶ pfu/eye or 1.5×10⁸ vp/eye) of Ad5 McEwen (Stock P3, Feb. 25, 05, 5.3×10⁸ pfu/ml). Eyes were closed and gently rubbed for 5 seconds to ensure contact of the virus on all ocular surfaces.

Eyes were cultured for virus after at least 3 hours (Day 0) after infection. Following topical anesthesia with proparacaine, a single cotton-tipped swab was placed into the lower fornix of each eye, rolled over the cornea into the upper fornix to recover adenovirus from the tear film and corneal and conjunctival surfaces. The swabs from each eye were placed individually into tubes containing 1 ml of outgrowth media and were frozen at −70° C. pending plaque assay. On Day 1, rabbits were divided into 3 treatment groups of 5 rabbits each. Table 2 provides the dosage regimen for this experiment. Two masked solutions were received. One solution contained 100 mg/ml of IG (Gammagard S/D) and one solution contained pharmaceutical grade saline for use as the negative control. The positive antiviral control, 0.5% cidofovir was not masked because of the differences in treatment regimen. The solutions were stored at 4° C. until and during use.

TABLE 2
dosage regimen.

| | | | N | n | Rabbit |
|---|---|---|---|---|---|
| Group | Drug | Treatment Regimen | Rabbits | Eyes | Numbers |
| I | NSR** | 4 times daily | 5 | 10 | 1-5 |
| | | for 10 days | | | |
| II | 0.5% | 2 times daily | 5 | 10 | 6-10 |
| | Cidofovir* | for 7 days | | | |
| III | NPR** | 4 times daily | 5 | 10 | 11-15 |
| | | for 10 days | | | |

*Total dose of Cidofovir was 2.6 mg. Drop size in all groups was 37 μl.
**See below.

Drops were administered with at least a 1 hour interval between drops. All eyes from all groups were cultured for virus on days 1, 3, 4, 5, 7, 9, 11, and 14 at least one hour after the final doses of the treatments described above. At various times during the course of the experiment, Ad5 titers were determined on A549 cell monolayers using a standard plaque assay. The ocular cultures to be titered were thawed, diluted (1:10) and inoculated onto A549 monolayers. The virus was adsorbed for 3 hours. Following adsorption, 1 ml of media plus 0.5% methylcellulose was added to each well, and the plates were incubated at 37° C. in a 5% CO₂-water vapor atmosphere. After 7 days incubation, the cells were stained with 0.5% gentian violet, and the number of plaques were counted under a dissecting microscope (25×). The viral titers were then calculated, and expressed as plaque-forming units per milliliter (pfu/ml).

Key to Experimental Drugs:

NSR: NSR was instilled using a Rainin EDP electronic pipet set in the multi-dispense mode. 37 μl drops were instilled. The solution was stored at 4° C. until and during use. After the code was broken, NSR was found to be 100 mg/ml IG.

Cidofovir: 6.75 ml of the 0.5% Cidofovir was prepared (5 mg/ml×6.75 ml=33.75 mg total of Cidofovir needed). 450 μl (33.75 mg) of the 75 mg/ml Vistide (Cidofovir Injection, Gilead Sciences Inc., Foster City, Calif. Lot #A301A1, Exp. 04/2006) was added to 6.3 ml of 0.9% Sodium Chloride Injection USP (Baxter Healthcare Corp. Deerfield, Ill.) to yield the 6.75 ml of 0.5% Cidofovir. Vistide was purchased from the pharmacy at the University of Pittsburgh Medical Center. The Cidofovir was instilled using a Rainin EDP electronic pipet set in the multi-dispense mode. 37 μl drops were instilled. The drug was stored at 4° C. during the study.

NPR: The NPR was instilled using a Rainin EDP electronic pipet set in the multi-dispense mode. 37 μl drops were instilled. The solution was stored at 4° C. until and during use. After the code was broken, NPR was found to be the Saline Control.

Tables 3-5 provide the raw data for viral ocular titers on different days during the study. Ocular viral titers from IG, Cidofovir and Normal saline treated animals were shown in table 3, 4 and 5 respectively.

TABLE 3
NSR (IG)-Ad-R1 Results

| Eye | D 0 | D 1 | D 3 | D 4 | D 5 | D 7 | D 9 | D 11 | D 14 |
|---|---|---|---|---|---|---|---|---|---|
| 1L | 450 | 15 | 70 | 5 | 200 | 0 | 0 | 0 | 0 |
| 1R | 1350 | 25 | 750 | 85 | 450 | 0 | 0 | 0 | 0 |
| 2L | 1350 | 90 | 250 | 40 | 450 | 0 | 0 | 0 | 0 |
| 2R | 17400 | 600 | 2600 | 5750 | 650 | 0 | 0 | 0 | 0 |
| 3L | 5400 | 2150 | 200 | 450 | 1400 | 0 | 0 | 0 | 0 |
| 3R | 14600 | 600 | 2300 | 90 | 605 | 0 | 0 | 0 | 0 |
| 4L | 5500 | 800 | 150 | 2500 | 150 | 5 | 0 | 0 | 0 |
| 4R | 6200 | 60 | 70 | 250 | 1300 | 15 | 0 | 0 | 0 |
| 5L | 2900 | 350 | 450 | 55 | 550 | 0 | 0 | 0 | 0 |
| 5R | 6850 | 350 | 1800 | 15 | 50 | 0 | 0 | 0 | 0 |

TABLE 4
0.5% Cidofovir (CDV) -Ad-R1 Results

| Eye | D 0 | D 1 | D 3 | D 4 | D 5 | D 7 | D 9 | D 11 | D 14 |
|---|---|---|---|---|---|---|---|---|---|
| 6L | 6650 | 1550 | 8200 | 2350 | 1400 | 15 | 0 | 0 | 0 |
| 6R | 7200 | 1800 | 9300 | 1800 | 1100 | 0 | 0 | 0 | 0 |
| 7L | 3100 | 330 | 100 | 25 | 0 | 0 | 0 | 0 | 0 |
| 7R | 2150 | 550 | 350 | 0 | 0 | 0 | 0 | 0 | 0 |
| 8L | 1040 | 450 | 210 | 80 | 5 | 0 | 0 | 0 | 0 |
| 8R | 2450 | 450 | 4250 | 650 | 550 | 0 | 0 | 0 | 0 |
| 9L | 1950 | 475 | 3800 | 400 | 10 | 0 | 0 | 0 | 0 |
| 9R | 4250 | 1000 | 2700 | 6700 | 1550 | 0 | 0 | 0 | 0 |
| 10L | 1100 | 5 | 130 | 1150 | 0 | 0 | 0 | 0 | 0 |
| 10R | 2250 | 550 | 9350 | 4700 | 400 | 5 | 0 | 0 | 0 |

TABLE 5
NPR (saline control)-Ad-R1 Results

| Eye | D 0 | D 1 | D 3 | D 4 | D 5 | D 7 | D 9 | D 11 | D 14 |
|---|---|---|---|---|---|---|---|---|---|
| 11L | 195 | 1150 | 5650 | 1550 | 1050 | 10 | 0 | 0 | 0 |
| 11R | 1300 | 600 | 6750 | 3450 | 730 | 160 | 0 | 0 | 0 |
| 12L | 8100 | 700 | 100 | 40 | 0 | 25 | 5 | 0 | 0 |
| 12R | 1700 | 300 | 1050 | 245 | 75 | 100 | 0 | 0 | 0 |
| 13L | 4350 | 1350 | 750 | 5450 | 210 | 5 | 0 | 0 | 0 |
| 13R | 1100 | 4000 | 2700 | 6100 | 1000 | 0 | 0 | 0 | 0 |
| 14L | 270 | 1450 | 2400 | 1050 | 25 | 5 | 0 | 0 | 0 |
| 14R | 9400 | 4650 | 6600 | 7050 | 2850 | 45 | 0 | 0 | 0 |
| 15L | 6450 | 1350 | 6250 | 1850 | 1050 | 10 | 0 | 0 | 0 |
| 15R | 7850 | 3100 | 6500 | 2350 | 735 | 50 | 5 | 0 | 0 |

Analysis of Results: Data were analyzed statistically using True Epistat and/or Minitab statistical software. Outcome measures included: Ad5-Positive Cultures per Total (Overall [Days 1-14]; Early Phase of Infection [Days 1-5]; Late Phase of Infection [Days 7-14]), Daily Viral Titers, Mean Viral Titer (Early Phase of Infection [Days 1-5]; Late Phase of Infection [Days 7-14]), and Duration of Shedding. Significance was established at the p≦0.05 confidence level.

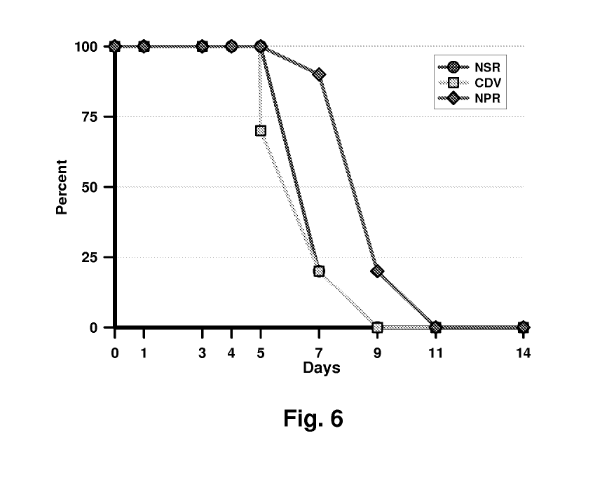

Ad5-Positive Cultures per Total: Table 6 and FIG. 6 summarize the results of number of positive cultures per total during the study period (day 1-14 post infection). In each treatment group, 10 eyes were swabbed for plaque assay as described previously. A total of 80 swabs (10×8 times) were taken from each group and result summarized as shown in Table 6. The overall number of positive culture per total was expressed as a percentage of the total. In this study 52.5%, 47.5% and 62.5% of IG (NSR), Cidofovir (CDV) and Normal Saline (NPR) treated animals positive cultures were seen respectively. The difference in this outcome was not statistically significant as shown by Chi-square analysis.

TABLE 6
IG-Ad-R1 Results - Positive Cultures per Total (Days 1-14)

| | Day* | | | | | | | | |
|---|---|---|---|---|---|---|---|---|---|
| Group | 1 | 3 | 4 | 5 | 7 | 9 | 11 | 14 | Total (%) |
| NSR | 10/10 | 10/10 | 10/10 | 10/10 | 2/10 | 0/10 | 0/10 | 0/10 | 42/80 |
| | 100% | 100% | 100% | 100% | 20% | 0% | 0% | 0% | 52.5% |
| 0.5% Cidofovir | 10/10 | 10/10 | 9/10 | 7/10 | 2/10 | 0/10 | 0/10 | 0/10 | 38/80 |
| (CDV) | 100% | 100% | 90% | 70% | 20% | 0% | 0% | 0% | 47.5% |
| NPR | 10/10 | 10/10 | 10/10 | 9/10 | 9/10 | 2/10 | 0/10 | 0/10 | 50/80 |
| | 100% | 100% | 100% | 90% | 90% | 20% | 0% | 0% | 62.5% |

*Day 0
NSR 10/10 (100%)
0.5% Cidofovir 10/10 (100%)
NPR 10/10 (100%)

Chi-Square Test: Positive Cultures/Total Days 1-14; expected counts* are provided below observed counts.** Chi-Sq=0.041+0.048+0.656+0.776+1.026+1.212=3.759; DF=2, P-Value=0.153 NS

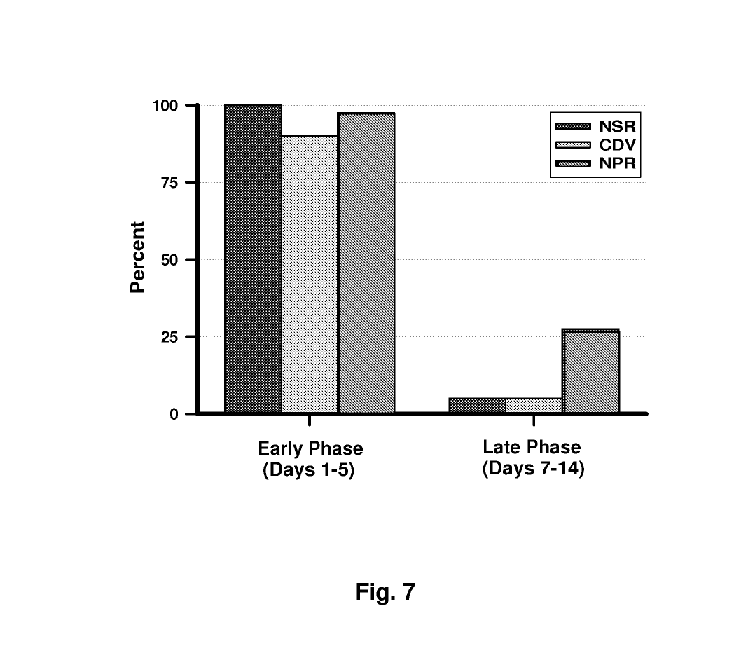

This analysis was further divided into early phase (day 1-5) and late Phase (day 7-14) as shown in Tables 7A and 7B. In the early phase, Chi-Square analysis shows that there was no significant difference among the three treatment groups. However in the late phase, both Cidofovir and IG treated animals had only 5% positive culture compared to 27.5% positive culture in the Normal saline treated group. The ratio of positive cultures per total in the late phase of the infection as presented in Table 7B was statistically analyzed using the Chi-Square test. Overall, there was similar significant difference between IG (NSR)/Cidofovir and Normal saline (NPR) (p=0.006). See FIG. 7.

TABLE 7A
Positive Cultures per Total (Days 1-5) (Early Phase of Infection)

| | Day | | | | |
|---|---|---|---|---|---|
| Group | 1 | 3 | 4 | 5 | Total (%) |
| NSR | 10/10 | 10/10 | 10/10 | 10/10 | 40/40 |
| | 100% | 100% | 100% | 100% | 100% |
| 0.5% Cidofovir | 10/10 | 10/10 | 9/10 | 7/10 | 36/40 |
| (CDV) | 100% | 100% | 90% | 70% | 90% |
| NPR | 10/10 | 10/10 | 10/10 | 9/10 | 39/40 |
| | 100% | 100% | 100% | 90% | 97.5% |

Chi-Square Test; Positive Cultures/Total Days 1-5, expected counts** are printed below observed counts*. Chi-Sq=0.072+1.667+0.142+3.267+0.012+0.267=5.426; DF=2, P-Value=0.066 NS. Three cells had expected counts less than 5.0. There was no significant statistical difference between the three groups

TABLE 7B
Positive Cultures per Total (Days 7-14) (Late Phase of Infection)

| | Day | | | | |
|---|---|---|---|---|---|
| Group | 7 | 9 | 11 | 14 | Total (%) |
| NSR | 2/10 | 0/10 | 0/10 | 0/10 | 2/40 |
| | 20% | 0% | 0% | 0% | 5% |
| 0.5% Cidofovir | 2/10 | 0/10 | 0/10 | 0/10 | 2/40 |
| (CDV) | 20% | 0% | 0% | 0% | 5% |
| NPR | 9/10 | 2/10 | 0/10 | 0/10 | 11/40 |
| | 90% | 20% | 0% | 0% | 27.5% |

Chi-Square Test: Positive Cultures/Total Days 7-14. Chi-Sq=1.800+0.257+1.800+0.257+7.200+1.029=12.343; DF=2, P-Value=0.002

Duration Of Viral Shedding

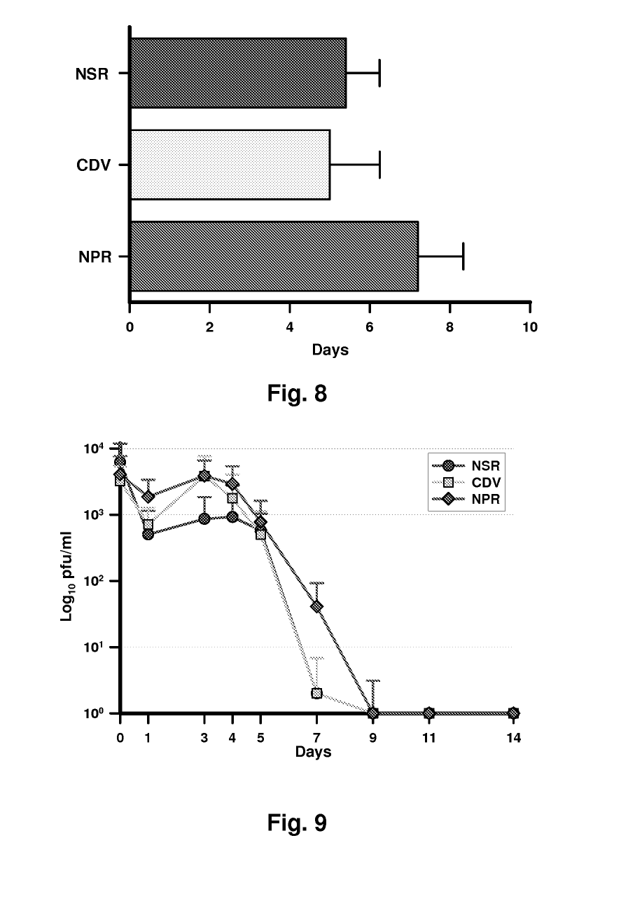

The duration of Ad5 shedding was determined for each eye by discerning the final day on which a positive Ad5 culture was detected, (that did not have three or more negative culture days between positive culture days) and calculating the mean and standard deviation for each treatment group). Duration of viral shedding is shown in Table 8 and was statistically analyzed. The duration of viral shedding in IG treated animals was 5.4 days (standard deviation of 0.84 days). In the Cidofovir treated animals, the duration of viral shedding was 5.0 (SD=1.25 days) while it was longer in the normal saline treated group: 7.2 days (SD=1.14 days). The significant finding is represented graphically in FIG. 8. In this analysis, IG was similar to Cidofovir in reducing the duration of viral shedding.

TABLE 8
Duration of Shedding

| NSREye | NSRDur | CDVEye | CDVDur | NPREye | NPRDur |
|---|---|---|---|---|---|
| 1L | 5 | 6L | 7 | 11L | 7 |
| 1R | 5 | 6R | 5 | 11R | 7 |
| 2L | 5 | 7L | 4 | 12L | 9 |
| 2R | 5 | 7R | 3 | 12R | 7 |
| 3L | 5 | 8L | 5 | 13L | 7 |
| 3R | 5 | 8R | 5 | 13R | 5 |
| 4L | 7 | 9L | 5 | 14L | 7 |
| 4R | 7 | 9R | 5 | 14R | 7 |
| 5L | 5 | 10L | 4 | 15L | 7 |
| 5R | 5 | 10R | 7 | 15R | 9 |

Mean Daily Ocular Titer

Mean daily ocular viral titers were obtained for IG treated animals (Table 9A), Cidofovir treated animals (Table 9B) and Normal saline treated group (Table 9C).

TABLE 9A
Daily Ad5 ocular titers, descriptive statistics, NSR

| Variable | N | Mean | Median | TrMean | StDev | SE Mean |
|---|---|---|---|---|---|---|
| NSR D0 | 10 | 6200 | 5450 | 5519 | 5662 | 1791 |
| NSR D1 | 10 | 504 | 350 | 359 | 641 | 203 |
| NSR D3 | 10 | 864 | 350 | 746 | 985 | 311 |
| NSR D4 | 10 | 924 | 87 | 436 | 1858 | 587 |
| NSR D5 | 10 | 580 | 500 | 544 | 452 | 143 |
| NSR D7 | 10 | 2.00 | 0.00 | 0.62 | 4.83 | 1.53 |
| NSR D9 | 10 | 0.00000 | 0.00000 | 0.00000 | 0.00000 | 0.00000 |
| NSR D11 | 10 | 0.00000 | 0.00000 | 0.00000 | 0.00000 | 0.00000 |
| NSR D14 | 10 | 0.00000 | 0.00000 | 0.00000 | 0.00000 | 0.00000 |

TABLE 9B
Descriptive Statistics, Cidofovir

| Variable | N | Mean | Median | TrMean | StDev | SEMean |
|---|---|---|---|---|---|---|
| CDVD0 | 10 | 3214 | 2350 | 2988 | 2166 | 685 |
| CDVD1 | 10 | 716 | 512 | 669 | 564 | 178 |
| CDVD3 | 10 | 3839 | 3250 | 3617 | 3855 | 1219 |
| CDVD4 | 10 | 1785 | 900 | 1394 | 2255 | 713 |
| CDVD5 | 10 | 501 | 205 | 433 | 625 | 198 |
| CDVD7 | 10 | 2.00 | 0.00 | 0.62 | 4.83 | 1.53 |
| CDVD9 | 10 | 0.00000 | 0.00000 | 0.00000 | 0.00000 | 0.00000 |
| CDVD11 | 10 | 0.00000 | 0.00000 | 0.00000 | 0.00000 | 0.00000 |
| CDVD14 | 10 | 0.00000 | 0.00000 | 0.00000 | 0.00000 | 0.00000 |

TABLE 9C
Descriptive Statistics, NPR

| Variable | N | Mean | Median | TrMean | StDev | SEMean |
|---|---|---|---|---|---|---|
| NPRD0 | 10 | 4072 | 3025 | 3890 | 3594 | 1137 |
| NPRD1 | 10 | 1865 | 1350 | 1713 | 1508 | 477 |
| NPRD3 | 10 | 3875 | 4175 | 3987 | 2727 | 862 |
| NPRD4 | 10 | 2913 | 2100 | 2756 | 2498 | 790 |
| NPRD5 | 10 | 773 | 733 | 609 | 849 | 268 |
| NPRD7 | 10 | 41.0 | 17.5 | 31.2 | 51.8 | 16.4 |
| NPRD9 | 10 | 1.000 | 0.000 | 0.625 | 2.108 | 0.667 |
| NPRD11 | 10 | 0.00000 | 0.00000 | 0.00000 | 0.00000 | 0.00000 |
| NPRD14 | 10 | 0.00000 | 0.00000 | 0.00000 | 0.00000 | 0.00000 |

The findings on different days were analyzed statistically. On day 0, there was no significant difference in the three groups. Following treatment on day 1, Viral titers of animals treated with IG was statistically significantly lower than the normal saline control. (p=0.011). It was also lower than Cidofovir treated animals, but not statistically significant. On Day 3, Iv-IgG treated group demonstrated statistically significant lower ocular titers compared to Cidofovir and Normal saline control. On Day 4, IG Treated animals had lower but not statistically significant ocular titers compared to Cidofovir and normal saline. On Day 5, there was no marked difference in viral ocular titers in the three groups. On Day 7, IG-treated animals had similar viral titers to Cidofovir-treated animals. Both drugs demonstrated significant lower ocular titers compared to normal saline. On day 9, both Cidofovir and IG treated animals were virus-free. Some normal saline treated animals still have ocular viral titers. There was no viral ocular titer on day 11 and 14 in the three groups. See FIG. 9.

Mean Combined Ocular Titers

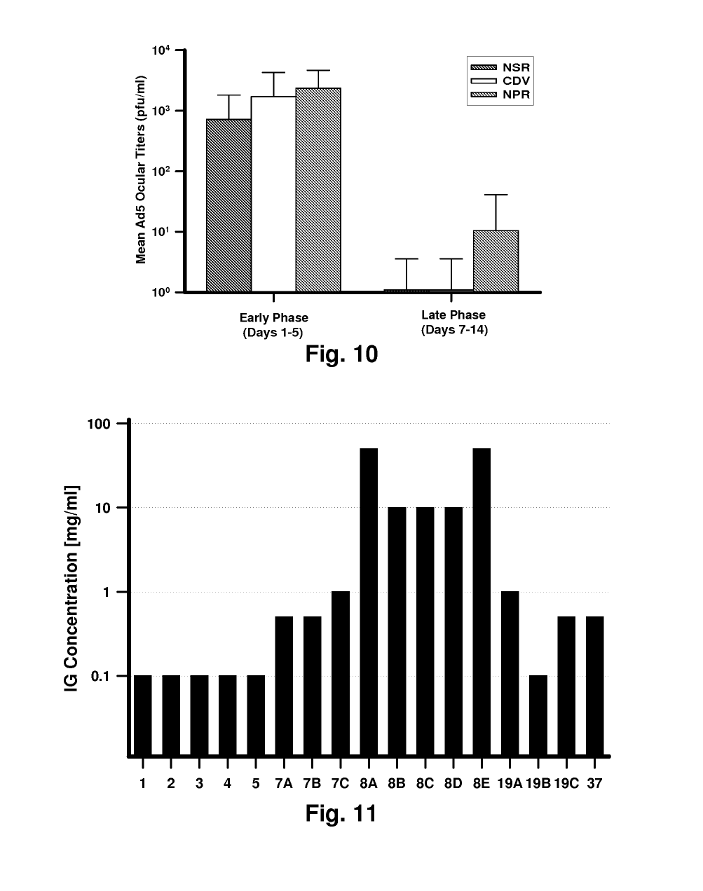

Mean Combined ocular viral titers were divided into early (day 1-5) and late phase (day 7-4) to study the antiviral properties of the three agents during active viral replication (early phase) and reduced viral replication (late phase). Statistical analysis shows that in the early phase of the disease, IG was more potent than Cidofovir and normal saline. There were significant lower combined mean ocular viral titers in animals treated with IG compared to Cidofovir and Saline (p=0.003). See FIG. 10. In the late phase of the infection, both IG and Cidofovir demonstrated similar significant lower combined ocular titer compared with Saline control (p=0.017). See FIG. 10.

Summary of Statistical Results

As stated above, NSR=100 mg/ml IG and NPR=Saline Control.

Positive Cultures per Total Days 1-14: The outcome measure for days 1-14 was the most stringent measure of global adenovirus replication over the entire course of the experiment since one virus plaque constitutes a positive culture. A total of 80 swabs (10×8 times) were taken from each group and result summarized as shown in Table 6. The overall number of positive culture per total was expressed as a percentage of the total. In this study 52.5%, 47.5% and 62.5% of IG (NSR), Cidofovir (CDV) and Normal Saline (NPR) treated animals positive cultures were seen respectively. The difference in this outcome was not statistically significant.

Positive Cultures per Total Days 1-5: This outcome measure determines the antiviral effect during the early phase of infection, during which the majority of acute viral replication takes place. There were no significant differences demonstrated between the three groups.

Positive Cultures per Total Days 7-14: This outcome measure determines the antiviral effect during the late phase of infection after the cessation of antiviral therapy and during which the immune and antiviral aided viral clearance takes place. NSR and 0.5% Cidofovir demonstrated significantly fewer Positive Cultures per Total Days 7-14 compared with NPR.

Mean Duration of Shedding: This outcome measure determines the mean length of the infection. NSR and 0.5% Cidofovir demonstrated significantly shorter durations of shedding compared with the NPR. There was no significant difference between NSR and 0.5% Cidofovir.

Mean Daily Ocular Titers: These outcome measures determine the viral replication on each day of the experiment. NSR and 0.5% Cidofovir demonstrated significantly lower titers on Days 1 and 7 compared with NPR. NSR demonstrated a significantly lower titer on Day 3 compared with 0.5% Cidofovir and NPR. There were no significant differences among any of the groups on any other day.

Mean Combined Ocular Titers Days 1-5: This outcome measure assesses the global viral replication during the Early Phase of Infection during which the majority of acute viral replication takes place. NSR demonstrated significantly a lower Mean Combined Ocular Titer Days 1-5 compared with 0.5% Cidofovir and NPR.

Mean Combined Ocular Titers Days 7-14: This outcome measure assesses the global viral replication during the Late Phase of Infection after the cessation of antiviral therapy and during which the immune and antiviral aided viral clearance takes place. NSR and 0.5% Cidofovir demonstrated significantly lower Mean Combined Ocular Titers Days 7-14 compared with NPR. There was no significant difference between NSR and 0.5% Cidofovir.

From these data and statistical analysis, the following conclusions can be made: 1) 100 mg/ml IG demonstrated antiviral efficacy in the Ad5/NZW rabbit ocular model as compared to the Saline control based on the outcome parameters of Positive Cultures per Total Days 7-14, Duration of Shedding, Mean Ocular Titers (Days 1, 3, 7), Mean Combined Ocular Titers Days 1-5 and Mean Combined Ocular Titers Days 7-14; 2) 0.5% cidofovir demonstrated its proven antiviral efficacy compared with the normal saline control group based on the outcome parameters of Positive Cultures per Total Days 7-14, Duration of Shedding, Mean Ocular Titers (Days 1, 7), and Mean Combined Ocular Titers Days 7-14; and 3) IG was comparable in antiviral activity with cidofovir, even demonstrating greater efficacy than cidofovir in reducing Ad5 titers on Day 3 and Combined Ocular Titers Days 1-5.

These results demonstrate the potential and efficacy of pooled, non-directed polyclonal antibody preparations, such as IG, in treatment of ocular infections. Because pooled, non-directed polyclonal antibody preparations have proven broad spectrum antimicrobial properties, including, for example and without limitation, against bacterial, viral, protozoa and fungal infections, it is expected, based on these data, that they will also be useful in treatment of ocular infections caused by a broad spectrum of microbial pathogens. Likewise, use of directed binding reagent preparations (e.g. polyclonal and monoclonal antibody preparations raised against specific pathogens), might also be expected to be useful in treating ocular infections.

### Example 4

### Additional Analysis of Ad Isolates

Log Reduction Neutralization—This study was conducted with multiple human ocular isolates of adenoviral serotypes 1, 2, 3, 4, 5, 7, 8, 19 and ATCC type Ad37. Test viral final concentration of 1×10⁶ plaque forming units per milliliter (pfu/ml) was incubated with IG final concentration of 1000, 500, 100, 10 and 1.0 μg/ml. Further experiments were conducted with IG final concentrations of 50, 10, 5, 1, and 0.1 mg/ml and 80, 50, 10, 5, and 1 mg/ml. After mixing, the viral/IG mixture was incubated for 1 hour at 37° C. in a water bath followed by 10 fold serial dilutions of each mixture. Samples were subjected to standard plaque assay using A549 cells, as described below. An IG concentration that demonstrated at least a 1 Log₁₀ decrease in Ad titer was considered to have significant antiviral activity. Results are provided in Table 10 and in FIG. 11.

TABLE 10
1log₁₀ Reduction of Ocular Isolates
of Adenoviral Serotypes By IG

| ISOLATES | IG (mg/ml) |
|---|---|
| Ad1 | 0.1 |
| Ad2 | 0.1 |
| Ad3 | 0.1 |
| Ad4 | 0.1 |
| Ad5 | 0.1 |
| Ad7A* | 0.5 |
| Ad7B | 0.5 |
| Ad7C | 1 |
| Ad8A | 50 |
| Ad8B | 10 |
| Ad8C | 10 |
| Ad8D | 10 |
| Ad8E | 50 |
| Ad19A | 1 |
| Ad19B | 0.1 |
| Ad19C | 0.5 |
| Ad37ATCC | 0.5 |

*individual isolates are identified by letter codes, for example Ad8 isolates were obtained from patients A-E, therefore Ad8 isolates are referred to as Ad8A-Ad8E.

### Example 5

### In Vivo Study, Part II

Additional experiments were conducted substantially as described in Example 3, in order to determine the antiviral efficacy of a 100 mg/ml solution of IG (intravenous Immune Globulin) in the Ad5/NZW rabbit ocular model. Raw data is provided in Tables 11A through 11c.

TABLE 11A
IG-Ad-R3 Results, (Underlined values
Determine Duration of Shedding)

| Eye | D 0 | D 1 | D 3 | D 4 | D 5 | D 7 | D 9 | D 11 | D 14 |
|---|---|---|---|---|---|---|---|---|---|
| 1L | 20 | 300 | 750 | 500 | 2700 | 65 | 10 | 0 | 0 |
| 1R | 250 | 0 | 7800 | 450 | 2850 | 620 | 130 | 15 | 0 |
| 2L | 1950 | 5 | 80 | 0 | 300 | 0 | 0 | 0 | 0 |
| 2R | 11000 | 0 | 1650 | 1800 | 2050 | 35 | 0 | 0 | 0 |
| 3L | 65 | 5 | 5200 | 1550 | 4650 | 630 | 150 | 0 | 0 |
| 3R | 250 | 450 | 4550 | 700 | 3050 | 90 | 0 | 0 | 0 |
| 4L | 550 | 35 | 1750 | 550 | 700 | 130 | 0 | 0 | 0 |
| 4R | 1100 | 25 | 1550 | 750 | 500 | 0 | 0 | 0 | 0 |
| 5L | 1500 | 0 | 550 | 5 | 0 | 30 | 0 | 0 | 0 |
| 5R | 90 | 20 | 1700 | 650 | 550 | 95 | 0 | 0 | 0 |

TABLE 11B
IG-Ad-R3 Results, 0.5% Cidofovir (Underlined
values Determine Duration of Shedding)

| Eye | D 0 | D 1 | D 3 | D 4 | D 5 | D 7 | D 9 | D 11 | D 14 |
|---|---|---|---|---|---|---|---|---|---|
| 6L | 20 | 200 | 2900 | 1150 | 700 | 25 | 0 | 0 | 0 |
| 6R | 600 | 700 | 1600 | 7100 | 600 | 5 | 0 | 0 | 0 |
| 7L | 750 | 400 | 4500 | 2000 | 850 | 5 | 0 | 0 | 0 |
| 7R | 140 | 700 | 3500 | 1900 | 950 | 10 | 0 | 0 | 0 |
| 8L | 1050 | 35 | 1500 | 0 | 15 | 0 | 0 | 0 | 0 |
| 8R | 50 | 10 | 5 | 5 | 0 | 0 | 0 | 0 | 0 |
| 9L | 1350 | 550 | 2900 | 750 | 750 | 0 | 0 | 0 | 0 |
| 9R | 1200 | 5 | 105 | 0 | 0 | 0 | 0 | 0 | 0 |
| 10L | 30 | 500 | 2600 | 1100 | 5 | 0 | 0 | 0 | 0 |
| 10R | 6300 | 60 | 1350 | 400 | 1400 | 0 | 0 | 0 | 0 |

TABLE 11C
IG-Ad-R3 Results, Saline Control (Underlined
values Determine Duration of Shedding)

| Eye | D 0 | D 1 | D 3 | D 4 | D 5 | D 7 | D 9 | D 11 | D 14 |
|---|---|---|---|---|---|---|---|---|---|
| 11L | 650 | 300 | 3450 | 1150 | 200 | 5 | 10 | 0 | 0 |
| 11R | 160 | 65 | 255 | 1500 | 650 | 100 | 125 | 0 | 0 |
| 12L | 300 | 250 | 3400 | 3450 | 9450 | 1560 | 2400 | 15 | 0 |
| 12R | 3500 | 900 | 6800 | 4000 | 6500 | 320 | 5 | 0 | 0 |
| 13L | 600 | 1050 | 8550 | 2100 | 4550 | 435 | 30 | 0 | 0 |
| 13R | 650 | 650 | 2500 | 2000 | 2600 | 85 | 35 | 0 | 0 |
| 14L | 65 | 50 | 1350 | 50 | 15 | 0 | 170 | 0 | 0 |
| 14R | 700 | 5 | 1700 | 400 | 1150 | 300 | 0 | 0 | 0 |
| 15L | 3900 | 350 | 8050 | 3450 | 350 | 5 | 15 | 0 | 0 |
| 15R | 1350 | 1200 | 6750 | 450 | 1550 | 1260 | 420 | 0 | 0 |

Raw Data is summarized in Tables 12A-12C. Tables 12B and 12C provide summaries extracted from Table 12A for early- and late-phase results.

TABLE 12A
Positive Cultures per Total (Days 1-14)

| | Day | | | | | | | | |
|---|---|---|---|---|---|---|---|---|---|
| Group | 1 | 3 | 4 | 5 | 7 | 9 | 11 | 14 | Total (%) |
| IG | 7/10 | 10/10 | 9/10 | 9/10 | 8/10 | 3/10 | 1/10 | 0/10 | 47/80 |
| | 70% | 100% | 90% | 90% | 80% | 30% | 10% | 0% | 58.75% |
| 0.5% Cidofovir | 10/10 | 10/10 | 8/10 | 8/10 | 4/10 | 0/10 | 0/10 | 0/10 | 40/80 |
| (CDV) | 100% | 100% | 80% | 80% | 40% | 0% | 0% | 0% | 50% |
| Saline Control | 10/10 | 10/10 | 10/10 | 10/10 | 9/10 | 9/10 | 1/10 | 0/10 | 59/80 |
| | 100% | 100% | 100% | 100% | 90% | 90% | 10% | 0% | 73.75% |

Day 0
IG 10/10 (100%)
0.5% Cidofovir 10/10 (100%)
Saline Control 10/10 (100%)

TABLE 12C
Positive Cultures per Total (Days 1-5) (Early Phase of Infection)

| | Day | | | | |
|---|---|---|---|---|---|
| Group | 1 | 3 | 4 | 5 | Total (%) |
| IG | 7/10 | 10/10 | 9/10 | 9/10 | 35/40 |
| | 70% | 100% | 90% | 90% | 87.5% |
| 0.5% Cidofovir | 10/10 | 10/10 | 8/10 | 8/10 | 36/40 |
| (CDV) | 100% | 100% | 80% | 80% | 90% |
| Saline Control | 10/10 | 10/10 | 10/10 | 10/10 | 40/40 |
| | 100% | 100% | 100% | 100% | 100% |

TABLE 12C
Positive Cultures per Total (Days 7-14) (Late Phase of Infection)

Day

| Group | 7 | 9 | 11 | 14 | Total (%) |
|---|---|---|---|---|---|
| IG | 8/10 | 3/10 | 1/10 | 0/10 | 12/40 |
| | 80% | 30% | 10% | 0% | 30% |
| 0.5% Cidofovir | 4/10 | 0/10 | 0/10 | 0/10 | 4/40 |
| (CDV) | 80% | 0% | 0% | 0% | 10% |
| Saline Control | 9/10 | 9/10 | 1/10 | 0/10 | 19/40 |
| | 90% | 90% | 10% | 0% | 47.5% |

Summary of Significant Results

Positive Cultures per Total Days 1-14—(This outcome measure is the most stringent measure of global adenovirus replication over the entire course of the experiment since one virus plaque constitutes a positive culture.) 10% IG and 0.5% Cidofovir demonstrated significantly fewer Positive Cultures per Total Days 1-14 compared with the saline Control. There was no difference between and IG and 0.5% Cidofovir.

Positive Cultures per Total Days 1-5—(This outcome measure determines the antiviral effect during the Early Phase of Infection during which the majority of acute viral replication takes place.) There were no significant differences demonstrated.

Positive Cultures per Total Days 7-14—(This outcome measure determines the antiviral effect during the Late Phase of Infection after the cessation of antiviral therapy and during which the immune and antiviral aided viral clearance takes place.) 0.5% Cidofovir demonstrated significantly fewer Positive Cultures per Total Days 7-14 compared with 10% IG and the saline Control. There was no difference between and IG and the saline Control.

Mean Duration of Shedding—(This outcome measure determines the mean length of the infection.) 10% IG and 0.5% Cidofovir demonstrated significantly shorter mean durations of shedding compared with the saline Control. 0.5% Cidofovir demonstrated a significantly shorter mean duration of shedding compared with 10% IG.

Mean Daily Ocular Titers—(These outcome measures determine the viral replication on each day of the experiment.) 10% IG demonstrated significantly lower titers on Day 1 compared with the saline Control. There were no significant difference between 10% IG and 0.5% Cidofovir on Days 1. 0.5% Cidofovir demonstrated a significantly lower titer on Day 7 compared the saline Control. There were no significant difference between 0.5% Cidofovir and 10% IG on Day 7. There were no significant differences among any of the groups on any other day.

Mean Combined Ocular Titers Days 1-5—(This outcome measure assesses the global viral replication during the Early Phase of Infection during which the majority of acute viral replication takes place.) 10% IG and 0.5% Cidofovir demonstrated significantly lower Mean Combined Ocular Titers Days 1-5 compared with the saline Control. There was no significant difference between 10% IG and 0.5% Cidofovir.

Mean Combined Ocular Titers Days 7-14—(This outcome measure assesses the global viral replication during the Late Phase of Infection after the cessation of antiviral therapy and during which the immune and antiviral aided viral clearance takes place.) 10% IG and 0.5% Cidofovir demonstrated significantly lower Mean Combined Ocular Titers Days 7-14 compared with the saline Control. There was no significant difference between 10% IG and 0.5% Cidofovir.

Conclusions

100 mg/ml IG demonstrated antiviral efficacy in the Ad5/NZW rabbit ocular model compared with the Saline control based on the outcome parameters of Positive Cultures per Total Days 1-14 and Days 7-14, Duration of Shedding, Mean Ocular Titers (Day 1), Mean Combined Ocular Titers Days 1-5 and Mean Combined Ocular Titers Days 7-14.

0.5% Cidofovir demonstrated its proven antiviral efficacy compared with the control group based on the outcome parameters of Positive Cultures per Total Days 1-14 and Days 7-14, Duration of Shedding, Mean Ocular Titers (Day 7), Mean Combined Ocular Titers Days 1-5, and Mean Combined Ocular Titers Days 7-14.

10% IG was comparable in antiviral activity with 0.5% Cidofovir is some outcome measures while it was less efficacious in others.

The results of this study suggest that future studies involving IG as a treatment for adenovirus ocular infections are warranted.

### Example 6

### Combined Analysis of Data Provided in Examples 3 and 5

The following summarizes the in vivo studies provided in Examples 3 and 5. Data from the two studies were combined, analyzed by Minitab statistical software using Analysis of Variance (ANOVA), and X² analyses. Significance was established at the p<0.05 confidence.

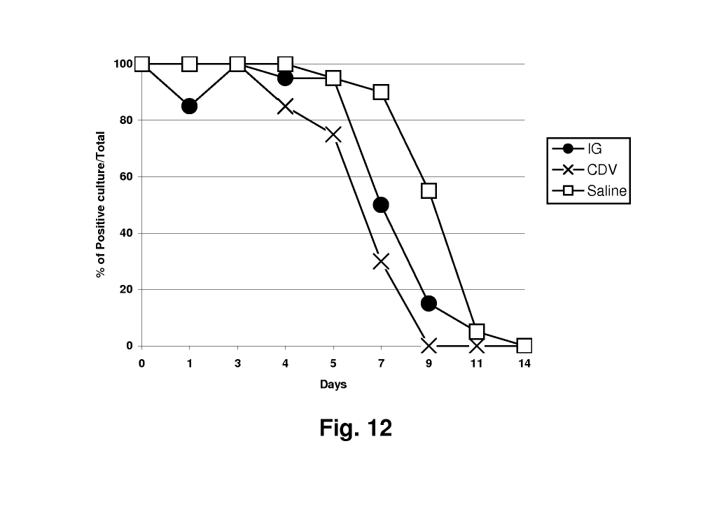

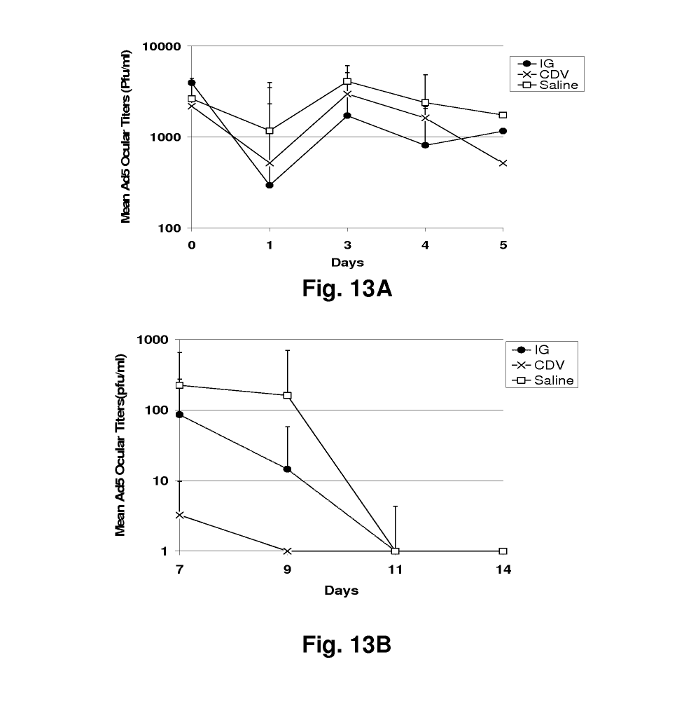

The results of the combined studies are summarized in Table 13, and FIGS. 12 and 13. The number of Ad5-positive cultures per total was determined for each treatment group by ascertaining the number of eye swabs that demonstrated a positive Ad5 culture per total number of cultures. These data were divided into the early (Days 1-5) phase of infection during which most of the adenovirus replication takes place and late (Days 7-14) phase of infection during which the normal immune and antiviral aided clearance of adenovirus occurs. A comparison of the total number of Ad5 positive cultures per total number of cultures taken per group over the entire course of the study (Days 1-14) demonstrated that both IG and 0.5% cidofovir demonstrated significant decreases in the number of Ad5 positive cultures per total (Table 2) compared with the control. Breaking these data down into the early (Days 1-5) and late (Days 7-14) phases of infection (Table 13), IG and cidofovir demonstrated significant antiviral reduction in number of positive cultures per total compared with the control during only the late phase of infection compared with the control. The daily reduction in percent Ad5 positive cultures per total for all treatment groups is presented graphically in FIG. 4. IG and cidofovir demonstrated significant decreases in the number of Ad5 positive cultures per total on Days 7 and 9 compared with the saline treated eyes.

TABLE 13
Viral Outcome Measures of 100 mg/ml IG
in the Ad5/NZW Rabbit Ocular Model.

| | | |
|---|---|---|
| IG (100 mg/ml) | Cidofovir (0·5%) | Saline |

Adenoviral Positive Culture/total

| | | | |
|---|---|---|---|
| Overall (Days 1-14) | 89/160 (55.6%)* | 78/160 (48.75%)* | 109/160 (68.1%) |
| Early Phase (Days 1-5) | 75/80 (93.75%) | 72/80 (90.0%) | 79/80 (98.75% |
| Late Phase (Days 7-14) | 14/80 (17.5%)* | 6/80 (7.5%)* | 30/80 (37.5%) |

Mean Combined Ad5 titer (pfu/ml)

| | | | |
|---|---|---|---|
| Early Phase (Days 1-5) | 9.9 ± 14.6 × 10²* | 1.4 ± 2.1 × 10³* | 2.3 ± 2.4 × 10³ |
| Mean ± Sd | | | |
| (Power 0·7599) (n = 80) | | | |
| Late Phase (Days 7-14) | 2.5 ± 10.1 × 10¹* | 0.8 ± 0.4 × 10⁰* | 9.6 ± 35.0 × 10¹ |
| Mean ± Sd | | | |
| (Power 0·9998) (n = 80) | | | |

Duration of Ad5 Shedding (Days)

| | | | |
|---|---|---|---|
| Mean ± Sd | 6.4 ± 1.7* | 5.3 ± 1.3* | 8.1 ± 1.4 |
| (Power 0·9998) (n = 20) | | | |

*P ≦ 0.05 when compared with the Control. Chi-Square was used for the analysis of Ad5-Positive Cultures/Total. ANOVA was used for the analysis of Mean Combined Ad5 Titer and Duration of Ad5 Shedding.

The mean combined Ad5 ocular titers represent a global measure of adenovirus replication during the early and late phases of infection. These were determined by calculating the mean and standard deviation of all ocular cultures from each treatment group during the early and late phases (n=80 for all groups). The results are presented in Table 13. During the early phase of infection, the mean Ad5 ocular titers were significantly decreased when the eyes were treated with IG and Cidofovir (p=0.0001, power 0.7599, ANOVA). Similar results were demonstrated during the late phase compared with the control (p=0.013, power 0.9998, ANOVA).

The mean duration of shedding was estimated by determining the last day on which adenovirus positive cultures were obtained and calculating the mean and standard deviation. The results, shown in Table 13, demonstrate that both IG and Cidofovir significantly decreased the duration of shedding compared with the saline control (p=0.008, power 0.9998, ANOVA). Furthermore, Cidofovir significantly decreased the Mean Duration of shedding compared with IG.

In general, IG and cidofovir demonstrated equivalent antiviral inhibitory activity (except for the duration of Ad5 shedding for which cidofovir was superior), and each antiviral was significantly better than the control group for the outcome measures described above.

In this study, we showed that IG met the previously suggested minimal criteria for development of an antiviral for the treatment of ocular adenoviral infections: namely: 1) antiviral activity against a wide range of adenovirus serotypes that infect the eye; 2) antiviral efficacy in the Ad5/NZW rabbit ocular model; and 3) safety following topical administration (Romanowski E G, Yates K A, Teuchner B, Nagl M, Irschick E U, Gordon Y J. N-Chlorotaurine is an effective antiviral agent against adenovirus in vitro and in the Ad5/NZW rabbit ocular model. Invest Ophthalmol Vis Sci. 2006;47:2021-2026).

In general, IG demonstrated antiviral activity that was equivalent to cidofovir despite major differences in their mechanisms of inhibitory action. While cidofovir is a nucleoside analog that works intra-cellularly to block DNA replication, IG works by neutralization of free infectious virus on the ocular surface. IG was remarkably effective during the critical early phase of infection (Days 1-5) as demonstrated in the significant reduction of mean ocular daily titers on days 1, 3 and 4 (See FIG. 13). IG also reduced the combined ocular titers during the early phase of infection compared to both cidofovir and saline treatment groups (see Table 13). These findings support that the rapidly acting IG, acts through extra-cellular viral neutralization on the ocular surface. The clinical implications may be summarized as follows: firstly, topical IG may accelerate clearance of the virus from infected eyes leading to a more rapid cure. Secondly, because of rapid decreases in ocular titers in the early phase of the infection, transmission to susceptible hosts will be limited thereby, reducing local epidemics. Thirdly, the prophylactic use of topical IG in susceptibles may prevent additional clinical infections. While IG and cidofovir were equivalent for most outcome parameters, cidofovir did demonstrate a significantly shorter duration of viral shedding (See Table 13) presumably due to its intracellular-mediated adenoviral DNA polymerase blocking activities (Romanowski E G, Gordon Y J, Araullo-Cruz T, Yates K A, Kinchington, P R. The antiviral resistance and replication of cidofovir-resistant adenovirus variants in the New Zealand white Rabbit Ocular Model. Invest Ophthalmol Vis Sci. 2001; 42:1812-1815) and prolonged tissue half-life following rapid uptake into cells.

Since commercial IG is produced from serum pooled from many donors, the issue of product consistency needs to be addressed during future development of an ophthalmic topical antiviral. Nevertheless, data from the current in vitro studies indicate that different lots of IG demonstrated similar antiviral features indicating that anti-adenoviral activity is consistent from lot to lot (see Example 1 and FIG. 1). Future studies to test the antiviral activity of IG from different manufacturers may also prove to be informative.

In summary, the current experimental study represents the first study to successfully evaluate topical antiviral properties of immunoglobulin preparations against etiologic agents of adenoviral ocular diseases both in vitro and in vivo. Due to its many beneficial properties, a topical solution containing immunoglobulin, whether directed or non-directed, may provide anti-inflammatory, anti-immune as well as antiviral activity against EKC. Furthermore, because of its broad-spectrum antimicrobial properties, topical ocular application of pooled immunoglobulin, such as IG, may be effective against other viral and bacterial causes of conjunctivitis. The potential risk of transmission of infectious diseases has been minimized by current methods of producing IG. Also the risk of anaphylaxis is minimal because of presumed very low levels of ocular absorption. The topical ophthalmic use of pooled human immunoglobulin preparations, such as IG, may be of immense benefit in the ophthalmology units, pediatric units, community clinics and for public health globally. These potential benefits and our preclinical data support further studies with topical immunoglobulin preparations, such as IG.

### Example 7

### Ocular Toxicity

Ocular toxicity studies were conducted using four groups of two animals using the Draize method. All the animals tolerated the drugs at concentrations including 6 mg/mL, 30 mg/mL and 100 mg/ml, as well as the control group which received a 0.5% Albumin solution, all in water. The Maximum Mean Total Score (MMTS) scores were 0 for all groups and therefore all IG concentrations and the placebo (albumin) were considered Non-Irritating. Ophthalmic examinations during the study demonstrated no corneal involvement, conjunctival reddening, chemosis or discharge, or iritis. Data is provided in Table 14.

| | | | |
|---|---|---|---|
| Group | Day 1 | Day 3 | Day 5 |

| | | | | | | |
|---|---|---|---|---|---|---|
| 30 | mg/mL | 0.5 N | 1.0 | PN | 0.5 | N |
| 0.5% | Albumin | 0.5 N | 0 | N | 0 | N |
| 100 | mg/mL | 0 N | 0 | N | 0 | N |
| 6 | mg/mL | 0.5 N | 0.5 | N | 0.5 | N |

There was no toxicity associated with the four solutions tested in this study. The highest IG concentration tested in this study is suitable for therapeutic use.

### Example 8

### Neutralization of HSV1

Herpes Simplex type 1 is a common ocular pathogenic virus. The inhibition of this virus by IG was investigated in two epithelial cell lines. Similar to the experiment mentioned in example one, 25, 12.5, 6.25, 3.12, 1.56, 0.78, 0.39, 0.2, 0.1, 0.05, 0.02 mg/ml of IG was incubated with 10⁶ pfu of HSV1 encoding green florescent protein for 1 hour at 37° C. in duplicates. In two separate experiments, 10⁵ cells of freshly harvested A549 or Vero epithelial cell lines was seeded to the plates and incubated overnight at 37° C. Inhibition of cell infection by IG was analyzed by flow cytometery and cell quest software.

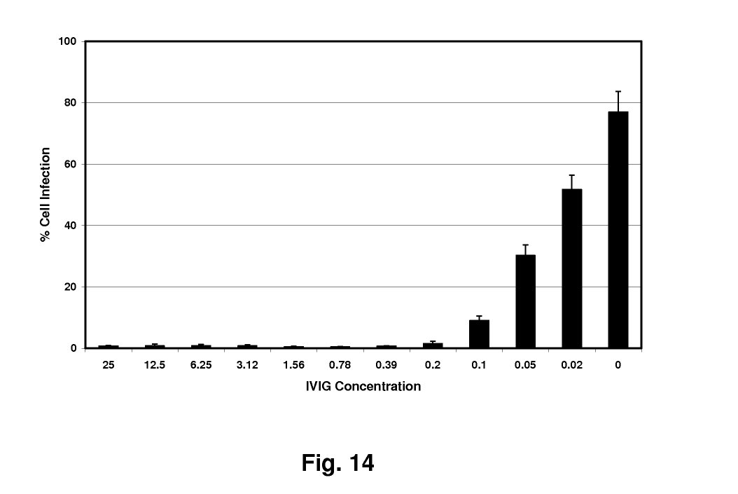

As shown in FIG. 14 and Table 15, less than 0.5 mg/ml of IG completely inhibited cell infection in both cell lines.

TABLE 15
Inhibition of HSV1 infection by IG

Percentage Cell Infection

| IG (mg/ml) | A549 Cell Line | Vero Cell Line | Mean/STD |
|---|---|---|---|
| 25 | 0.44 | 0.93 | 0.69 ± 0.3 |
| 12.5 | 0.20 | 1.36 | 0.78 ± 0.6 |
| 6.25 | 0.46 | 1.20 | 0.83 ± 0.4 |
| 3.12 | 0.61 | 1.18 | 0.90 ± 0.3 |
| 1.56 | 0.31 | 0.73 | 0.52 ± 0.2 |
| 0.78 | 0.43 | 0.54 | 0.49 ± 0.1 |
| 0.39 | 0.78 | 0.71 | 0.75 ± 0.0 |
| 0.2 | 2.27 | 0.82 | 1.55 ± 0.7 |
| 0.1 | 10.51 | 7.7 | 9.11 ± 1.4 |
| 0.05 | 33.66 | 26.72 | 30.19 ± 3.5 |
| 0.02 | 56.36 | 47.08 | 51.72 ± 4.6 |
| 0.00 | 83.61 | 70.45 | 77.03 ± 6.6 |

Having described this invention above, it will be understood to those of ordinary skill in the art that the same can be performed within a wide and equivalent range of conditions, formulations and other parameters without affecting the scope of the invention or any embodiment thereof.

We claim:

1. A method of preventing or treating an ocular infection caused by a viral, fungal, protozoa or bacterial agent in a patient comprising administering to an eye of the patient a composition comprising a binding reagent reactive against the viral, fungal, protozoa or bacterial agent in an amount effective to prevent or treat the infection.

2. The method of claim 1, wherein the binding reagent is an antibody.

3. The method of claim 2, wherein the antibody is a monoclonal antibody or a polyclonal antibody.

4. The method of claim 1, wherein the binding reagent is a human or humanized antibody.

5. The method of claim 1, wherein the composition comprising a binding reagent comprises human immunoglobulin.

6. The method of claim 5, wherein the composition comprising a binding reagent comprises a pooled human immunoglobulin preparation.

7. The method of claim 6, wherein between 1 μg and 100 mg of pooled human immunoglobulin is administered to the patient from one to ten times daily for treatment or prophylaxis.

8. The method of claim 7, wherein from about 10 μg to about 5 mg of pooled human immunoglobulin is administered from two to six times daily for one to 14 days.

9. The method of claim 1, wherein the composition comprising a binding reagent comprises a pooled human immunoglobulin preparation suitable for intravenous use.

10. The method of claim 9, wherein the pooled human human immunogloulin preparation is Gammagard S/D immune Globulin Intravenous (Human) (Baxter Healthcare Corporation).

11. The method of claim 1, wherein the viral, fungal, protozoa or bacterial agent is one of S. aureus, S. pneumoniae, H. influenzae, Neisseria gonorrhoeae and Chlamydia trachomatis, Adenovirus, Herpes Simplex, Herpes zoster virus, Candida species, Acanthamoeba species and Enteroviruses.

12. The method of claim 11, wherein the viral, fungal, protozoa or bacterial agent is adenovirus.

13. The method of claim 1, wherein an anti-inflammatory agent is administered while the binding reagent is administered to the patient.

14. The method of claim 13, wherein the anti-inflammatory agent is co-administered with the binding reagent in a single drug product.

15. The method of claim 13, wherein the anti-inflammatory agent is a non-steroidal anti-inflammatory agent.

16. The method of claim 15, wherein the non-steroidal anti-inflammatory agent is one of nepafenac, ketorolac, tromethamine, acetaminophen and bromfenac.

17. The method of claim 1, wherein an antibiotic is administered while the binding reagent is administered to the patient.

18. The method of claim 17, wherein the antibiotic is one or more of ciprofloxacin, norfloxacin, afloxacin, levofloxacin, gentamicin, tobramycin, neomycin, erythromycin, trimethoprim sulphate, and polymixin B.

19. The method of claim 1, wherein the binding reagent is administered in an opthamologically acceptable carrier.

20. The method of claim 19, wherein the opthamologically acceptable carrier is a liquid or hydrogel.

21. The method of claim 19, wherein the composition comprises one or more of CMC, PVP, a buffer, a rheology modifier, a buffer, and a chelating agent.

22. A composition comprising a binding reagent reactive against a viral, fungal, protozoa or bacterial agent and one or both of an antibiotic and an anti-inflammatory agent in amounts effective to treat an ocular infection by the viral, fungal, protozoa or bacterial agent, and an opthamologically acceptable carrier

23. The method of claim 22, wherein the composition comprises an anti-inflammatory agent that is a non-steroidal anti-inflammatory agent.

24. The method of claim 23, wherein the non-steroidal anti-inflammatory agent is one of nepafenac, ketorolac, tromethamine, acetaminophen and bromfenac.

25. The method of claim 22, wherein the composition comprises an antibiotic.

26. The method of claim 25, wherein the antibiotic is one or more of ciprofloxacin, norfloxacin, afloxacin, levofloxacin, gentamicin, tobramycin, neomycin, erythromycin, trimethoprim sulphate, and polymixin B.

27. A product comprising an opthamologically acceptable ocular dispenser containing a composition comprising a binding reagent reactive against a viral, fungal, protozoa or bacterial agent, in amounts effective to treat an ocular infection by the viral, fungal, protozoa or bacterial agent, in an opthamologically acceptable carrier.

28. The product of claim 27, wherein the opthamologically acceptable ocular dispenser is an eye-dropper.

29. The product of claim 27, wherein the binding reagent is an antibody.

30. The product of claim 29, wherein the antibody is a monoclonal antibody or a polyclonal antibody.

31. The product of claim 30, wherein the binding reagent is a human or humanized antibody.

32. The product of claim 27, wherein the composition comprising a binding reagent comprises a human immunoglobulin preparation.

33. The product of claim 32, wherein the composition comprising a binding reagent comprises a pooled human immunoglobulin preparation.

34. The product of claim 32, wherein the composition comprising a binding reagent comprises a pooled human immunoglobulin preparation suitable for intravenous use.

35. The product of claim 34, wherein the pooled human immunoglobulin preparation is Gammagard S/D immune Globulin Intravenous (Human) (Baxter Healthcare Corporation).

36. The product of claim 27, wherein the composition further comprises an anti-inflammatory agent.

37. The product of claim 37, wherein the anti-inflammatory agent is co-administered with the binding reagent.

38. The product of claim 37, wherein the anti-inflammatory agent is a non-steroidal anti-inflammatory agent.

39. The product of claim 39, wherein the non-steroidal anti-inflammatory agent is one of nepafenac, ketorolac, tromethamine, acetaminophen and bromfenac.

40. The product of claim 27, wherein the opthamologically acceptable carrier is a liquid or hydrogel.

41. The product of claim 27, wherein the composition comprises one or more of carboxymethylcellulose, polyvinylpyrrolidone, a buffer, a rheology modifier, a buffer, and a chelating agent.

42. The product of claim 27, the composition further comprises an antibiotic.

43. The product of claim 42, wherein the antibiotic is one or more of ciprofloxacin, norfloxacin, afloxacin, levofloxacin, gentamicin, tobramycin, neomycin, erythromycin, trimethoprim sulphate, and polymixin B.
